(19) United States
(12) Patent Application Publication
(10) Pub. No.: US 2007/0270822 A1
(43) Pub. Date: Nov. 22, 2007

(54) Bone fixation grommet

(75) Inventor: Eric S. Heinz, Memphis, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/413,525

(22) Filed: Apr. 28, 2006

(51) Int. Cl. A61F 2/30

(52) U.S. Cl. 606/61

(57) ABSTRACT

Embodiments of the invention include bone opening protection devices that may be deformed to match the size and contour of the bone. The deformation may occur by compression, expansion or resilient movement to an original configuration. A method is also provided for implantation of the bone opening protection devices in the spine.

### FIELD OF THE INVENTION

The present invention relates to bone fixation systems which use flexible members to lock two or more bone segments together, including the back, or spinous process of adjacent vertebrae or to attach a bone to another implanted device. More particularly, the invention relates to a grommet for protecting the bone from being injured by a tether or other connection member and methods of implantation.

### BACKGROUND

Severe back pain and nerve damage may be caused by injured, degraded, or diseased spinal joints and particularly, spinal discs. Current methods of treating these damaged spinal discs may include vertebral fusion, nucleus replacements, or motion preservation disc prostheses. Disc deterioration and other spinal deterioration may cause spinal stenosis, a narrowing of the spinal canal and/or the intervertebral foramen, that causes pinching of the spinal cord and associated nerves. In spinal surgery, the surgeon often locks adjacent vertebrae together. In some cases, the surgeon additionally locks adjacent vertebrae together through the spinous process by a cable around boney edges or through holes drilled in the bone. Alternative and potentially less invasive options are needed to provide spinal pain relief.

### SUMMARY

One embodiment of the invention is a bone opening protection device having at least one movable portion for anchoring to the bone.

In another aspect, the invention provides an apparatus for protecting bone surrounding an opening extending through bone from being injured by a connector. The apparatus comprising a body having an exterior surface and an interior surface defining a passage through the body from a first side to an opposite second side configured to receive the connector; an enlarged head engaged to the first side of the body and configured from engaging a first exterior portion of bone adjacent the opening. The body further includes an anchoring portion engaged to the second side of the body, the anchoring portion having an insertion configuration having a first external dimension and an anchoring configuration having a second external dimension, wherein the second external dimension is larger than the first external dimension. In one embodiment, the anchor portion is movable to the anchoring configuration by compression along the longitudinal axis of the body. In an alternative form, the anchor portion is movable to the anchoring configuration by inflation of a device associated with the anchor portion. In a further alternative form, the anchor portion returns to the anchoring position after removal of the insertion device.

In still a further aspect, an apparatus is provided for protecting bone surrounding an opening extending through bone from being injured by a connector. The apparatus comprising a body having an exterior surface and an interior surface defining a passage through the body from a first end portion to an opposite second end portion, the passage configured to receive the connector; and at least one of the first end portion or the second end portion including an anchoring portion having an insertion configuration having a first external dimension and an anchoring configuration having a second external dimension. The second external dimension is larger than the first external dimension and the anchoring portion is movable between the insertion configuration and the anchoring configuration.

In yet a further aspect, an apparatus is provided for lining a bone opening extending from a first side to an opposite second side. The apparatus comprises a bone anchor having a tubular body for insertion within the bone opening, a first end for engagement with the bone adjacent the first side and an opposite second end for engagement with the bone adjacent the opposite second side. The bone anchor having an insertion configuration with a first length extending between the first end and the opposite second end; and an anchoring configuration with a second length extending between the first end and the opposite second end. The first length is longer than the second length such that the bone anchor is movable from the insertion configuration to the anchoring configuration. In one aspect, at least the first end is obliquely oriented with respect to the longitudinal axis to substantially match the adjacent exterior bone surface. In another embodiment, the tubular body is in two pieces that are joined by telescopic engagement within the bone.

In a further aspect, a method is provided for implanting a bone protector in the spinous process. The method comprises providing a bone opening protector having a proximal end and an opposite distal end; inserting at least a portion of the bone opening protector from a first side of the spinous process towards the opposite side of the spinous process; and moving the distal end toward the proximal end to anchor the bone opening protector to the spinous process.

In another aspect, the present invention provides a method of placing a bone opening protector. The method includes the steps of forming an opening through a bone portion, inserting a bone opening protector and enlarging at least a portion of the bone opening protector to inhibit movement through the bone opening. In a further aspect, a spacing implant is placed with the bone opening protector and a tether is passed through the bone opening protector and attached to the spacing implant.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is a perspective view of the spine in combination with one embodiment of the present invention.

FIG. 2A is a side view of a grommet according to one aspect of the present invention.

FIG. 2B is a partial cross sectional side view of another embodiment of a grommet.

FIG. 2C is a partial cross sectional side view of still another embodiment of a grommet.

FIG. 2D is a side view of still a further embodiment of a grommet.

FIG. 2E is a partial cross sectional side view of a further grommet assembly.

FIG. 2F is a partial cross sectional side view of a bone protection device.

FIG. 2G is a partial cross sectional side view of a further bone protection device.

FIGS. 3A-3D illustrates the implantation of a grommet in accordance with one aspect of the present invention.

FIGS. 4A-4C illustrate the implantation of a grommet in accordance with another aspect of the present invention.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

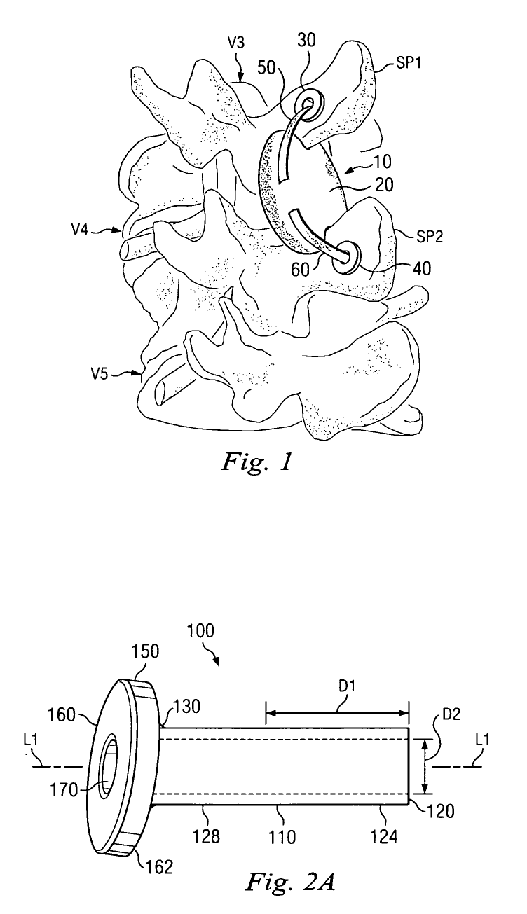

Referring now to FIG. 1, there is shown a segment of the spine with an implant system 10 extending between spinous processes SP1 and SP2. The implant system 10 includes an elastomeric block 20 disposed between the spinous processes SP1/SP2 held in position by tether members 50 and 60 extending through bone grommets 30 and 40, respectively. Various aspects about the implant system, bone grommets and methods for implantation will be further described below.

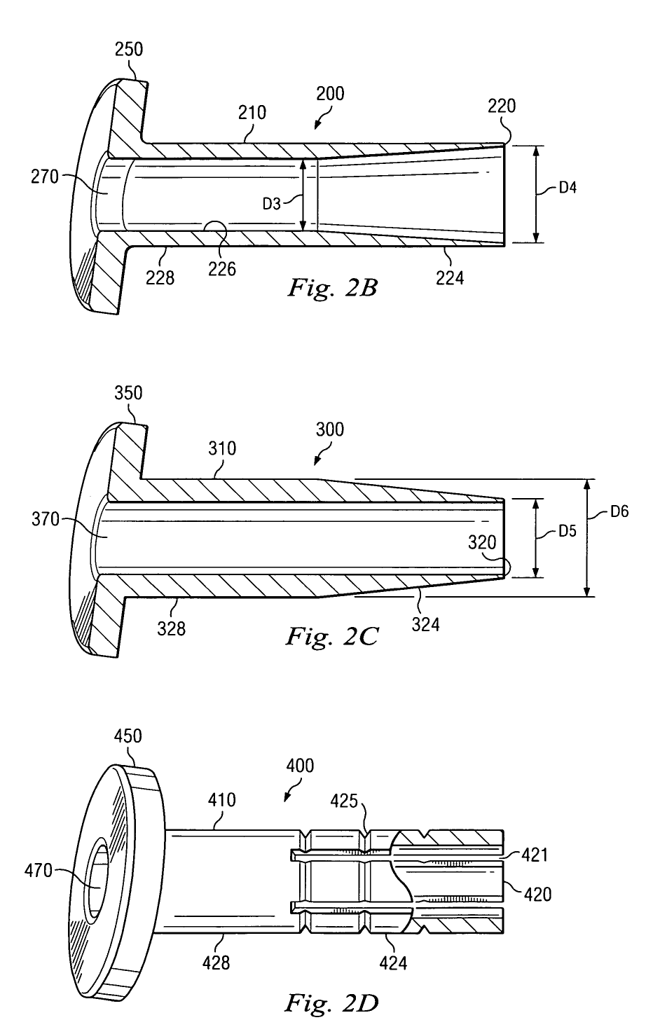

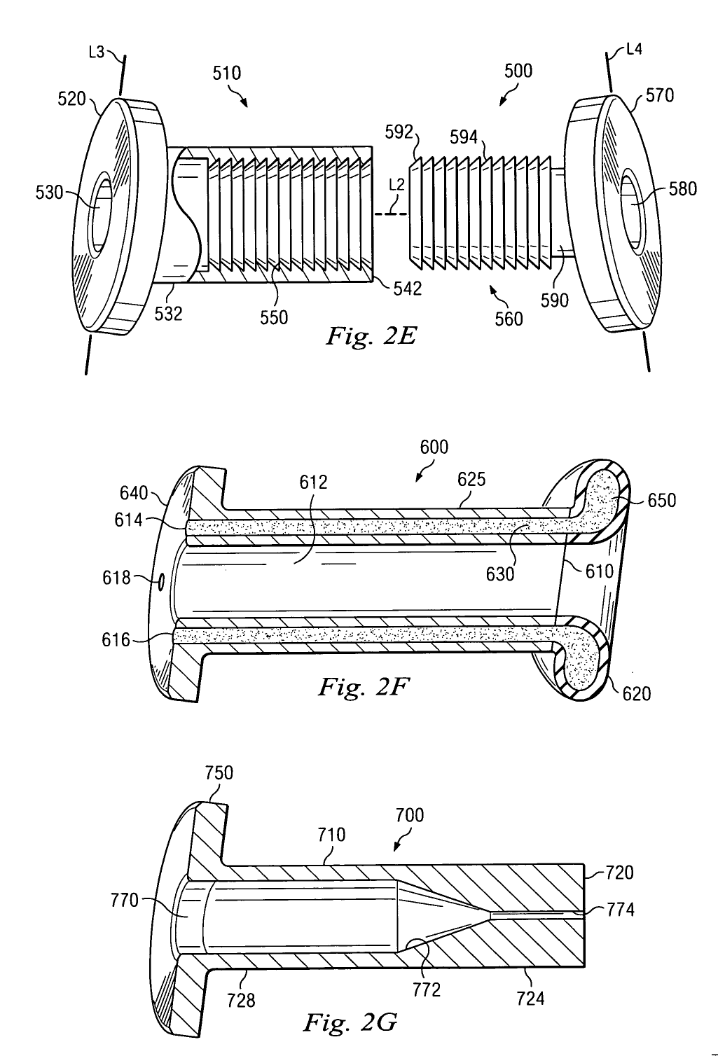

FIG. 2A illustrates an embodiment of a bone grommet 100 according to one aspect of the present invention. Grommet 100 includes a tubular body 110 having a leading end 120, a deformation portion 124 and a static portion 128. The tubular body transitions to a larger outside diameter over taper 130 as it joins to the head 150. Head 150 includes an exterior surface 160 and an opposite bone engaging surface 160. A central passage 170 extends along the longitudinal axis L1 through tubular body 110 providing a conduit between leading tip 120 and head 150. The central passage 170 has a substantially constant internal diameter D2. In the illustrated embodiment, the deformable portion 124 extends for a deformation length of D1 between leading tip 120 and static portion 128.

FIG. 2B illustrates a further embodiment of a grommet 200 according to an alternative aspect of the present invention. Grommet 200 includes a tubular body 210 having a leading end 220, a deformation portion 224 and a static portion 228. The tubular member 210 is integrally formed with head 250. The tubular body 210 has a substantially uniform outer diameter extending between tip and head 250. The internal diameter D3 of the passage 270 is substantially uniform through head 250 and in area 226 of static portion 228, but then begins to increase in diameter through the deformation portion 224 as it extends to leading end 220 to a final diameter of D4. The increase in the internal diameter of the passage through deformation portion 224 creates an area of decreasing wall thickness for tubular member 210. It will be appreciated that the deformation portion 224 will have a tendency to deform in the smallest wall thickness areas first to form an enlarged bone engagement head. Further, the opening of passage 270 to head 250 has a radiused or chamfered edge to inhibit stress on the connection member as it leaves the grommet.

Referring now to FIG. 2C, there is shown a further embodiment of a bone grommet according to another aspect of the present invention. Bone grommet 300 includes a tubular body 310 having a leading end 320, a deformation portion 324 and a static portion 328. The tubular member 310 is integrally formed with head 350. The tubular body 310 has a passage 370 with a substantially uniform inner diameter D5 extending between tip 320 and head 350. The external diameter D6 of the tubular member 310 is substantially uniform from head 350 through static portion 328, but then begins to decrease in diameter through the deformation portion 324 as it extends to leading end 320 and final diameter D5. The decrease in external diameter of the tubular member 310 through deformation portion 324 creates an area of decreasing wall thickness. It will be appreciated that the deformation portion 324 will have a tendency to deform in the smallest wall thickness areas first to form an enlarged bone engagement head. Further, the reduced external diameter may assist the user in advancing the grommet through bone during implantation.

A further embodiment of the present invention is shown in FIG. 2D. Bone grommet 400 includes a tubular body 410 having a leading end 420, a deformation portion 424 and a static portion 428. The tubular member 410 is integrally formed with head 450. The tubular body 410 has a passage 470 with a substantially uniform inner diameter extending between tip 420 and head 450. The wall thickness of tubular member 410 is substantially constant along its length. However, in the deformation portion 424, at least one and preferable four longitudinal slits 421 are formed in the side walls of the tubular member. In the illustrated embodiment, the slits 421 extend all the way through leading end 420 and from the exterior surface to the interior surface of passage 470. A series of grooves 425 are formed on the outer surface of the tubular member in the deformation area. In this manner, the fingers of tubular wall between slits 421 have stress relief areas defined by grooves 425 such that there is a tendency to deform in the area of the grooves. Further, in an alternative embodiment, the slits 421 do not extend through leading tip 420 such that there remains a circumferential portion of material joining the fingers near tip 420. In this embodiment, as force is applied along the longitudinal axis of the device, the middle area of the deformable portion 424 will tend to flex outwardly at the grooves to an enlarge configuration while the circumferential portion of material at the leading tip 420 maintains the original diameter. Further, while slits 421 have been illustrated as extending completely through the side wall of the tubular member, it is contemplated that in an alternative embodiment, there are longitudinal grooves rather than or in combination with slits. The material may be frangible along the longitudinal grooves or may have a greater flexibility at the grooves to allow easier expansion for more rigid materials.

Referring to FIG. 2E, there is shown still a further embodiment of a bone grommet according to the present invention. Bone grommet 500 includes a first member 510 and a mating second member 560. First member 510 includes an enlarged bone engaging head 520, a tubular member 532 having a central passage 530 extending there through and exiting head 520. Central passage 530 includes an internal surface having a plurality of ratchet teeth 550 projecting toward head 520 and away from leading end 542. The mating second member 560 includes a tubular member 590 and an enlarged bone engagement head 570. An internal passage 580 extends through tubular member 590 and exits through engagement head 570. A series of ratchet teeth 594 are formed on the exterior of tubular member 590 projecting toward head 570 and away from leading tip 592. The external diameter of tubular member 590 is configured to be a substantially close fit with the internal diameter of tubular member 532. As leading end 592 of tubular member 590 is advanced within passage 530 of tubular member 532 along longitudinal axis L2, the ratchet teeth 594 will slip past ratchet teeth 550. It will be appreciated that the engagement between ratchet teeth 550 and 594 will inhibit withdrawal of the tubular member 590 from passage 530. Further, head 520 has a bone mating surface and an opposite external surface oriented along axis L3 oblique to axis L2. In a similar manner, head 570 has a bone mating surface and an opposite external surface oriented along axis L4 oblique to axis L2 in a substantially congruent manner.

FIG. 2F illustrates a further embodiment of a bone protection device according to the present invention. Bone protector 600 includes an inner cannula 610 defining an internal passage 612. A balloon anchor 620 is disposed on the distal end of the cannula 610 and is shown in its extended, anchoring position in FIG. 2F. A flowable material 650 is transmitted from the bone engaging head 640 through one or more conduits 630 extending exterior to internal passage 612 and within protective sheath 625. An insertion tool, not shown, engages filling holes 514, 616, and 618 in head 640 to transmit filling material 650 into the device 600. In one aspect, the flowable material tends to create a relatively rigid structure within balloon 620. For example, the flowable material may be a bone cement or other curable compound.

In an alternative embodiment, a relative rigid structure surrounds at least a portion of balloon 620 such that the structure is deformed into an anchoring configuration as a result of the balloon being expanded. The balloon may be deflated and the relatively rigid structure will remain in its anchoring configuration after the balloon is deflated. In still a further embodiment, the balloon 620 is provided on a separate cannula. In this embodiment, the balloon is positioned in the internal passage adjacent the deformable portion of any of the grommets disclosed herein and expanded. The expansion of the balloon results in deformation of the deformable configuration into an anchoring position. The balloon may be deflated and removed after causing the grommet to be anchored against the bone.

Still a further embodiment of the present invention is shown in FIG. 2G. Bone protection device 700 includes a tubular body 710 extending between head 750 and leading end 720. Tubular body has a static portion 728 and a deformable portion 724 configured for movement into an anchoring position. The tubular body defines an internal passage 770 having a first diameter within the static portion 728, narrowing through tapered internal wall 772 to the narrow passage 774 having a second diameter. The first diameter is greater than the second diameter. The tapered internal wall 772 and the narrow passage 774 extend within the deformable portion 724. It will be appreciated that a conical wedge having a maximum diameter substantially equal to the first diameter will create significant expansion in the deformable portion 724 as it passes longitudinal along the axis of the device. After the expansion, the conical wedge may pass out of the protection device 700 by movement through passage 770.

In an alternative aspect, the each of the above described embodiments may be formed from one or more individual components. For example, the grommet may be provided as a substantially uniform tubular member. Under longitudinal compression, each end of the tube may deform to a bone engaging configuration. In a further aspect, at least one of the ends of the bone grommet is biased to return to the enlarged bone engaging configuration. During insertion, an instrument compresses the resilient end and after removal of the insertion instrument, the end returns to its enlarged bone engagement configuration. In one aspect, the grommet is formed of a shape memory alloy and one or both ends are compressed during insertion and return to their anchoring bone engagement configurations after the insertion instrument is withdrawn. Further, as a result of the expansion of the distal end and or compression along the longitudinal axis, the grommets of the present invention tend to be shorter longitudinally in their anchoring configurations than in their insertion configurations. Moreover, the longitudinal length in the anchoring configuration substantially matches the thickness or width of the spinous process adjacent the bone opening.

Preferably, the ends of the grommet have a radius leading into the central bore extending between opposite sides to minimize strain on the tether as it bends into the bore. Note that the grommet of the invention has been described as being generally cylindrical. The external and internal shapes of grommet have been shown for the purpose of illustration and may take any form such as, but without limitation, oval, square, triangular, etc., as the benefits of the invention may be found in any grommet which may be readily positioned and held in an opening formed in bone so long as the grommet may be inserted into the opening where it will stay in place and guide the cable to prevent sawing of the bone. Moreover, while the device has been shown as a circumferentially closed tubular member, it is contemplated that at least a portion of the longitudinally extending wall may be eliminated such that only the wall aligned with the direction of the compressive force applied by the anchoring tether must be included to obtain the benefits of the bone protection device.

Embodiments of the implant in whole or in part may be constructed of biocompatible materials of various types. Examples of implant materials include, but are not limited to, non-reinforced polymers, carbon-reinforced polymer composites, PEEK and PEEK composites, shape-memory alloys, titanium, titanium alloys, cobalt chrome alloys, stainless steel, ceramics and combinations thereof. If the instrument or implant is made from radiolucent material, radiographic markers can be located on the instruments or implant to provide the ability to monitor and determine radiographically or fluoroscopically the location of the body in the spinal space.

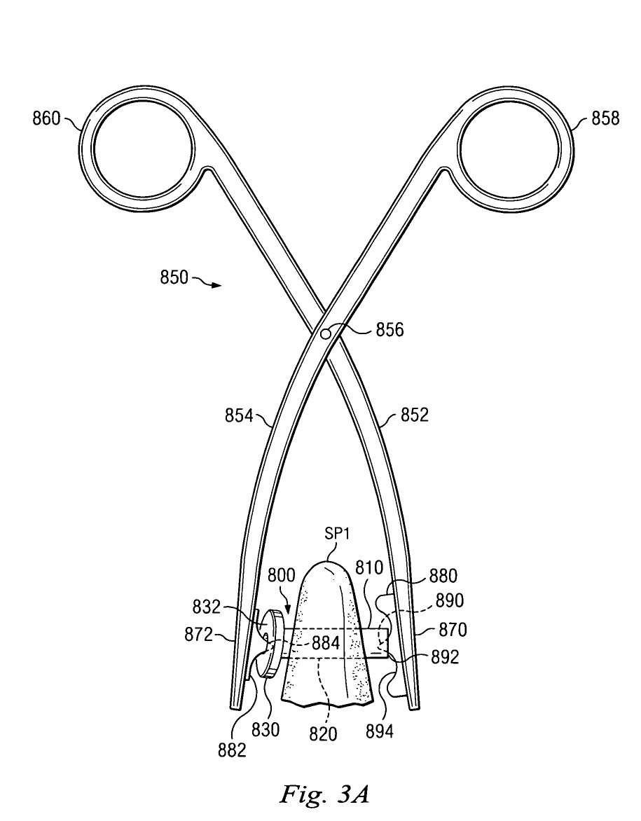

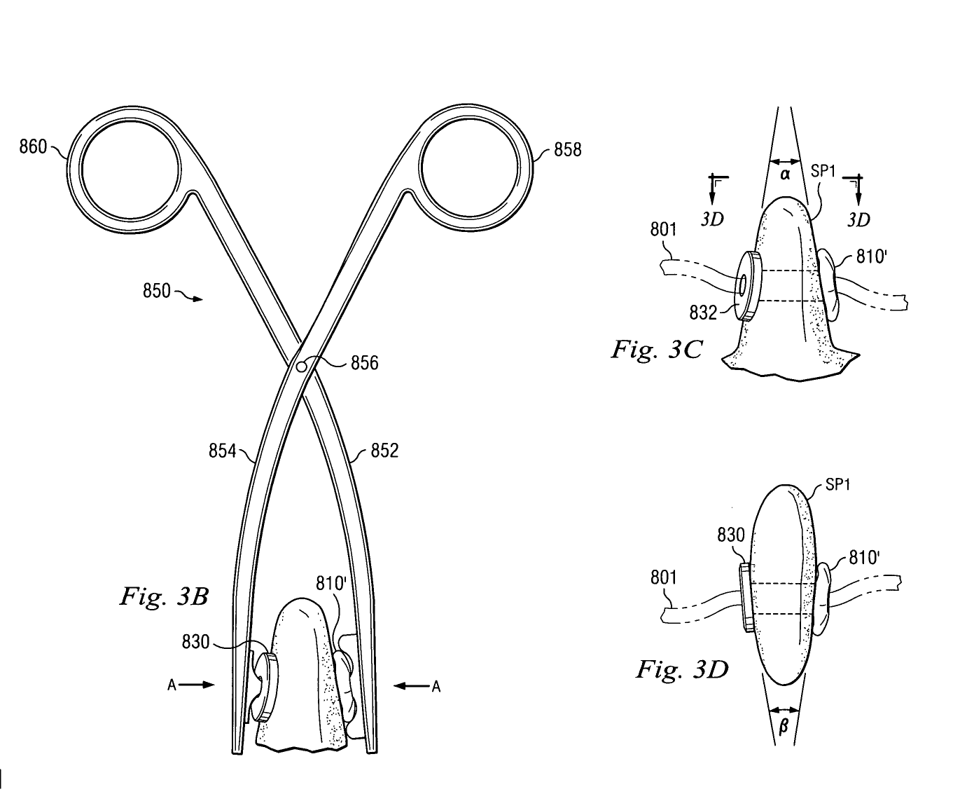

Referring now to FIGS. 3A-3D, there is illustrated a method for implantation of a bone grommet. A grommet 800 is positioned in a hole extending from a first lateral side of the spinous process SP1 to the opposite second lateral side. The enlarged head 830 of the grommet is engaged against first lateral side of the spinous process and the tubular static portion 820 extends within the bone of the spinous process. The deformable portion 810 extends distally beyond the second lateral side of the spinous process. In one form, the passage through the bone is formed by a separate instrument such as a drill, stylet, probe, awl or curette in advance of grommet insertion. The grommet is then inserted into the previously formed opening. In another embodiment, the grommet is positioned over a hole forming device, such as a guide wire or stylet, and positioned simultaneously with the formation of the opening through the bone.

Once the grommet 800 has been positioned from one side of the spinous process to the other, a deformation tool 850 is engaged. Tool 850 includes a pair of articulating branches 852 and 854 joined by pivot pin 856. Each branch 852, 854 includes a handle 860, 858, respectively, at the proximal end of the instrument. Adjacent the distal end 870 of branch 852, the tool includes a first die 880 interconnected with the tool. Die 880 includes an alignment projection 890, a tapering cone portion 892 and a flattened surface 894. The opposing branch 854 includes a die 882 attached to distal end 872. Die 882 has an alignment projection 884 and a substantially planar flattening face to engage head 830. Once alignment projection 884 and 890 are positioned within the internal passage of the grommet, handles 860 and 858 may be articulated to create compressive force between distal ends 870 and 872 as shown by arrows A. As shown in FIG. 3B, movement of the die 880 toward die 830 tends to move the deformable portion 810 of the grommet to conform to the shape of the die and the outer surface of the spinous process. A series of dies 880 and 882 may be provided with different orientations of the flattening portions to match the external geometry of the spinous process. Alternatively, dies 880 and 882 may be pivotally mounted on branches 870 and 872 to allow pivoting during use so the flattening surfaces can pivot to substantially match the orientation of the outer surface of the spinous process. Once the deformable portion has been moved to the anchoring position sufficient to maintain the position in the bone, tool 850 may be removed and a tether 801 may be positioned through the internal channel.

As shown in FIG. 3C, as seen from the longitudinal axis of the spine, head 830 and deformed head 810′ are angled toward one another by angle α. The axial angle α in the axial plane of the spine between the bone contacting surface of head 830 and deformed head 810′ is between 0 and 30 degrees, and more approximately 15 degrees. Referring now to FIG. 3D, a top view of the implanted device as seen from the posterior of the spine transverse to the longitudinal axis of the spine shows that the head 830 and deformed head 810′ are angled toward one another by angle β in this aspect as well. The coronal angle β in coronal plane of the spine between the bone engaging surface of the head 830 and the deformed head 810′ is between 0 and 30 degrees, and more approximately 15 degrees. Each of the bone engaging surface and exterior surface of 810′ extend at an oblique angle with respect to the longitudinal axis of the grommet 800. Surface 832 and the corresponding bone engaging surface of head 830 extend at a congruent oblique angle with respect to the longitudinal axis.

As will be appreciated, in one aspect the present invention allows the bone grommet to be deformed to substantially match the natural contours of the spinous process bone. These contours vary depending on the anterior to posterior location of the implantation along the spinous process as well as the inferior to superior location. Further, these bone contours change according to the level of the spine and the individual vertebrae. For example, in the axial plane of a cervical vertebra, the middle of the spinous process has the smallest width with the bone expanding in width towards the anterior spinal canal and expanding in width posteriorly towards the bifurcated apex of the spinous process. As disclosed herein with respect to several of the embodiments, the grommet can be configured or deformed to substantial match the complex contours of the spinous processes along the length of the spine. In the axial plane, particularly in the cervical spine, the grommet may have a first angulation anteriorly and a second, different angulation as it extends posteriorly from the implantation site. In addition, the grommet may also be angled in the coronal plane with respect to the longitudinal axis of the grommet.

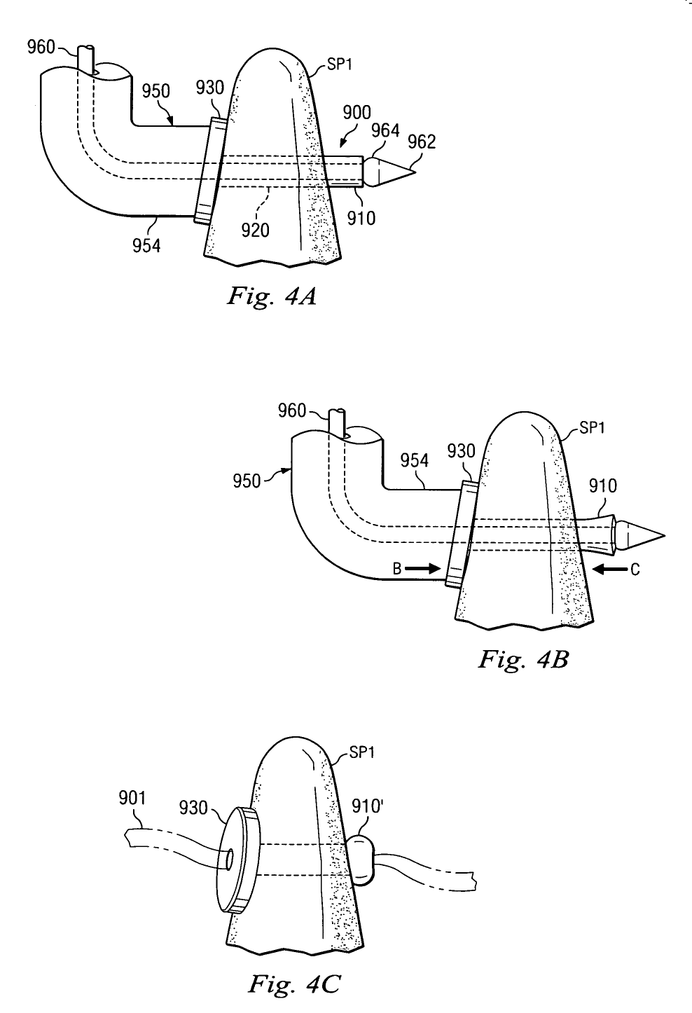

Referring now to FIG. 4A, there is shown a further embodiment of a grommet insertion tool according to another aspect of the present invention. Insertion tool 950 has an outer sleeve 954 and an inner member 960 extending within internal passage 952. Inner member 960 extends beyond the distal end of the outer sleeve 954. In the illustrated embodiment, the distal portion of tool 950 has a 90 degree offset. A grommet 900 may be positioned over the proximal portion of inner member 960 and advanced distally toward the distal end. The inner member is then inserted within outer sleeve 954 until the head 930 abuts the distal end of the outer sleeve. The distal end of inner member 960 has a pointed projection 962 adapted to pierce bone and a trailing end die 964. The die has a larger diameter than the deformable portion 910 of the grommet 900.

In one use, the spine is accessed from a unilateral posterior through a small incision or puncture wound in patient. In one aspect, the incision is large enough to access at least one level of the spine. A grommet 900 is positioned on the insertion device 950 as shown in FIG. 4A. The tool is advanced through the posterior incision until the tool is oriented as shown in FIG. 4A. The insertion tool tip 962 is positioned on a first lateral side of the spinous process and by applying force in the direction of arrow B is advanced through the bone to the opposite second lateral side of the spinous process. The grommet 900 is advanced with the tool as it forms the opening in the bone such that deformable portion 910 extends distally beyond the opposite lateral side and static portion 920 is positioned in the bone. In one embodiment, head 930 includes spikes to engage the bone. Once the head 930 had been firmly positioned to engage the bone on the first lateral side, the outer tube 954 is firmly held in position with force applied in the direction of arrow B while tension is applied to inner member 960 in the direction of arrow C. As inner member 960 moves proximally with respect to outer tube 954, the die 964 deforms the deformable portion 910 to an enlarged anchoring configuration larger in diameter than the opening in the bone. In the illustrated embodiment, the grommet 900 is formed substantially as shown with respect to FIG. 2G. Once the die 964 is withdrawn proximally past the deformable portion, the die can freely slide through the internal passage adjacent the static portion 920 having an inner diameter substantially matching the outer diameter of die 964. A similar grommet can be placed in the adjacent spinous process. A spacing implant is placed between the adjacent spinous processes through the incision. A first needle attached to a first connection member, such as a tether 901, is passed through the first lateral side portion of grommet 900 and out the opposite second lateral side. In one aspect, the needle is manipulated to engage the opposite side of the implant. A similar tether is placed through the grommet in the adjacent spinous process. Each of the tethers is tensioned to the desired level to hold the spacing implant in the desired position.

Use of the term “tether” herein is meant to cover cables, wires, tapes, bands, ropes, sutures, brackets and the like used in surgery to anchor an implant or tie together vertebrae. The tether may be metal or non-metal and is meant to fully cover any material used as a tether or tether equivalent. Further, the grommet may be coupled to the tether or a portion of a spacing implant prior to implantation in the bone. For example, when implanting the flexible spacer of FIG. 1, grommets 30 and 40 may be two piece devices similar to FIG. 2E. Each portion of the grommet is joined to the anchoring portion of the flexible spacer 20 prior to implantation. Thus, with the two pieces of the grommet joined to each other inside the bone, the spacer implant is anchored to the bone. This provides anchoring to the bone in a single step with placement of the grommet. In this embodiment, the grommet acts as both a protector for the bone opening and a bone anchor to secure the spacing implant to the bone.

Placement of the grommet may be in conjunction with placement of tissue materials including, but are not limited to, synthetic or natural autograft, allograft or xenograft, and may be resorbable or non-resorbable in nature. Examples of other tissue materials include, but are not limited to, hard tissues, connective tissues, demineralized bone matrix and combinations thereof. Examples of resorbable materials that may be used include, but are not limited to, polylactide, polyglycolide, tyrosine-derived polycarbonate, polyanhydride, polyorthoester, polyphosphazene, calcium phosphate, hydroxyapatite, bioactive glass, and combinations thereof. Implant may be solid, porous, spongy, perforated, drilled, and/or open.

Access to the surgical site may be through any surgical approach that will allow adequate visualization and/or manipulation of the skeletal structures. Example surgical approaches include, but are not limited to, any one or combination of anterior, antero-lateral, posterior, postero-lateral, transforaminal, and/or far lateral approaches. Implant insertion can occur through a single pathway or through multiple pathways, or through multiple pathways to multiple levels of the spinal column. Minimally invasive techniques employing instruments and implants are also contemplated. It is understood that all spatial references, such as “top,” “inner,” “outer,” “bottom,” “left,” “right,” “anterior,” “posterior,” “superior,” “inferior,” “medial,” “lateral,” “upper,” and “lower” are for illustrative purposes only and can be varied within the scope of the disclosure.

FIG. 1 illustrates portions of three vertebrae, V3-V5, of a typical lumbar spine. While embodiments of the invention may be applied to the lumbar spinal region, embodiments may also be applied to the cervical or thoracic spine or between other skeletal structures within the body.

While embodiments of the invention have been illustrated and described in detail in the disclosure, the disclosure is to be considered as illustrative and not restrictive in character. All changes and modifications that come within the spirit of the invention are to be considered within the scope of the disclosure.

What is claimed is:

1. An apparatus for protecting bone surrounding an opening extending through bone from being injured by a connector, the apparatus comprising:
a body having an exterior surface and an interior surface defining a passage through said body from a first side to an opposite second side, said passage configured to receive the connector;
an enlarged head connected to said first side of said body and configured from engaging a first exterior portion of bone adjacent the opening; and
an anchoring portion engaged to said second side of said body, said anchoring portion having an insertion configuration having a first external dimension and an anchoring configuration having a second external dimension, wherein said second external dimension is larger than said first external dimension, said anchoring portion expandable from the insertion configuration to the anchoring configuration.

2. The apparatus of claim 1, wherein said passage is at least partially cylindrical.

3. The apparatus of claim 2, wherein the passage is defined by a substantially continuous sidewall extending between open ends.

4. The apparatus of claim 1, wherein said anchor portion is formed of a deformable material and the anchoring portion is deformed at least in part when expanding from the insertion configuration and the anchoring configuration.

5. The apparatus of claim 1, wherein said anchoring portion is formed of a resilient material and the anchoring portion at least in part resiliently expands from the insertion configuration to the anchoring configuration.

6. The apparatus of claim 1, wherein the anchoring portion is formed of an inflatable portion and the anchoring portion at least in part inflates when expanding from the insertion configuration to the anchoring configuration.

7. An apparatus for protecting bone surrounding an opening extending through bone from being injured by a connector, the apparatus comprising:
a body having an exterior surface and an interior surface defining a passage through the body from a first end portion to an opposite second end portion, the passage configured to receive the connector; and
at least one of said first end portion or said second end portion including an anchoring portion having an insertion configuration having a first external dimension and an anchoring configuration having a second external dimension, wherein said second external dimension is larger than said first external dimension, said anchoring portion movable between the insertion configuration and the anchoring configuration.

8. The apparatus of claim 7, wherein both of said first end portion and said second end portion are movable between the insertion configuration and the anchoring configuration.

9. An apparatus for lining a bone opening extending from a first side to an opposite second side, comprising:
a bone anchor having a tubular body for insertion within the bone opening, the bone anchor having a first end for engagement with the bone adjacent the first side and an opposite second end for engagement with the bone adjacent the opposite second side,
said bone anchor having an insertion configuration with a first length extending between said first end and said opposite second end; said bone anchor having an anchoring configuration with a second length extending between said first end and said opposite second end, said first length longer than said second length;
wherein said bone anchor is movable from said insertion configuration to said anchoring configuration.

10. The apparatus of claim 9, wherein said tubular body is integrally formed with at least one of said first or second ends.

11. The apparatus of claim 9, wherein said first end is connected to a first tubular body portion and said second end is connected to a second tubular body portion, said first tubular body portion interconnected with said second tubular body portion to define said tubular body.

12. The apparatus of claim 11, wherein said first tubular body portion is telescopingly received within said second tubular body portion.

13. The apparatus of claim 9, wherein at least said first end is formed of a deformable material, said first end deformed at least in part to move from said insertion configuration to said anchoring configuration.

14. The apparatus of claim 9, wherein bone opening has a passage length extending from the first side to the second side, wherein said first length is substantially greater than said passage length and said second length substantially matches said passage length.

15. The apparatus of claim 9, wherein said tubular body has a longitudinal axis and at least said first end has an outer surface extending at an oblique angle with respect to said longitudinal axis.

16. A vertebral bone grommet for extending from a first outside bone surface to an opposite second outside bone surface, the grommet comprising:
a first bone anchoring assembly having a first surface configured for engagement with the first outside bone surface;
a second bone anchoring assembly having a second surface configured for engagement with the second outside bone surface; and
a connection assembly engaged between said first bone anchoring assembly and said second bone anchoring assembly.

17. The bone grommet of claim 16, wherein at least one of said first bone anchoring assembly or said second bone anchoring assembly includes an insertion configuration having a first external dimension and an anchoring configuration having a second external dimension, wherein said second external dimension is larger than said first external dimension, said anchoring portion expandable from the insertion configuration to the anchoring configuration.

18. The bone grommet of claim 16, wherein said connection assembly includes a first portion joined to the first bone anchoring assembly and a second portion joined to said second bone anchoring assembly, said first portion movable with respect to said second portion.

19. The bone grommet of claim 18, wherein said first portion is telescopingly received within said second portion.

20. The bone grommet of claim 19, wherein said first portion includes a retention surface engageable with said second portion to inhibit removal of the first portion from the second portion.

21. The bone grommet of claim 16, wherein the connection assembly has a longitudinal axis and said first surface extends at an oblique angle with respect to said longitudinal axis.

22. The bone grommet of claim 21, wherein said vertebral bone is a spinous process and said longitudinal axis extending substantially perpendicular to a patient's spinal axis, said oblique angle being between 0 to 30 degrees in the axial plane.

23. The bone grommet of claim 21, wherein said vertebral bone is a spinous process and said longitudinal axis extending substantially perpendicular to a patient's spinal axis, said oblique angle being between 0 to 30 degrees in the coronal plane.

24. The bone grommet of claim 23, wherein said first surface extends at a second oblique angle in the axial plane.

25. The bone grommet of claim 24, wherein said second surface extends at a corresponding congruent angle substantially matching said oblique angle and said second angle.

26. A method of implanting a bone opening protector, comprising:
forming an opening through a bone portion;
inserting a bone opening protector into the opening;
and moving at least a portion of the bone opening protector to change an external dimension to inhibit movement of the bone opening protector through the bone opening.

27. The method of claim 26, further including implanting a spacing element between a pair of vertebrae and attaching the spacing element to at least one vertebrae through the bone opening protector.

28. The method of claim 26, wherein said forming and said inserting are conducting in a single step by forcibly inserting the bone opening protector through the bone.

29. A method of implanting a bone protector in the spinous process, comprising:
providing a bone opening protector having a proximal end and an opposite distal end;
inserting at least a portion of the bone opening protector from a first side of the spinous process towards the opposite side of the spinous process; and
moving the distal end toward the proximal end to anchor the bone opening protector in the spinous process.

30. The method of claim 29, wherein said moving includes deforming said distal end outside of the spinous process.

31. The method of claim 29, wherein said moving includes expanding the distal end outside of the spinous process.

32. The method of claim 31, wherein said proximal end is connected to a proximal tubular body and said distal end is connected to a distal tubular body, said proximal tubular body and said distal tubular body are separate components, and said inserting includes inserting said proximal tubular body into the spinous process from a proximal side, and inserting said distal tubular body into the spinous process from a distal side.
